(19) United States
(12) Patent Application Publication
(10) Pub. No.: US 2010/0130788 A1
(43) Pub. Date: May 27, 2010

(54) METHOD FOR PRODUCING AMINES

(75) Inventors: Joana Coelho Tsou, Heidelberg (DE); Ekkehard Schwab, Neustadt (DE); Petr Kubanek, Mannheim (DE); Wolfgang Mackenroth, Tervuren (DE); Steffen Oehlenschlaeger, Antwerpen (BE); Hartwig Voss, Frankenthal (DE); Sameul Neto, Dresden (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, Ludwigshafen (DE)

(21) Appl. No.: 12/598,436

(22) PCT Filed: May 5, 2008
(86) PCT No.: PCT/EP08/55447

§ 371 Date: Nov. 2, 2009

(30) Foreign Application Priority Data
May 10, 2007 (EP) 07107893.5

(51) Int. Cl. C07C 209/36; B01J 21/18; B01J 23/52; B01J 23/44; B01J 23/755

(52) U.S. Cl. 564/422; 502/185; 502/184

(57) ABSTRACT

The invention relates to a process for preparing aromatic amines by catalytic hydrogenation of the corresponding nitro compounds, in particular for preparing toluenediamine by hydrogenation of dinitrotoluene, wherein hydrogenation catalysts in which a mixture of nickel, palladium and an additional element selected from the group consisting of cobalt, iron, vanadium, manganese, chromium, platinum, iridium, gold, bismuth, molybdenum, selenium, tellurium, tin and antimony is present as active component on a support are used.

The invention relates to a process for preparing amines by catalytic hydrogenation of the corresponding nitro compounds and also novel catalysts for carrying out the process.

The preparation of amines, in particular aromatic monoamines, diamines and/or polyamines, by catalytic hydrogenation of the corresponding mononitro, dinitro and/or polynitro compounds has been known for a long time and is widely described in the literature. An aromatic amine which is frequently used in industry is toluenediamine (TDA) which can be processed further to give tolylene diisocyanate and is prepared by hydrogenation of dinitrotoluene (DNT). A problem in the hydrogenation of DNT is the increased formation of by-products; apart from low boilers, usually deaminated and ring-hydrogenated products, relatively high molecular weight or tar-like products frequently occur and can lead not only to a reduction in the yield of the process but also to premature deactivation of the catalyst.

Hydrogenation catalysts used are, as described, for example, in EP-A-0 124 010, frequently metals of transition group VIII of the Periodic Table, in particular Raney iron, Raney cobalt and Raney nickel.

Catalysts comprising noble metals, in particular palladium but also platinum, are also frequently used for the hydrogenation of nitroaromatics. Catalysts comprising platinum and nickel are also known for this purpose.

Thus, U.S. Pat. No. 3,127,356 describes a process for producing hydrogenation catalysts which can be used for the hydrogenation of DNT to TDA. The catalysts comprise a support, an oleophilic carbon component such as carbon black, onto which the metals are applied. Here, the nickel is present as oxide or hydroxide in the catalyst.

U.S. Pat. No. 5,214,212 describes a process for the ring hydrogenation of aromatic amines. A noble metal catalyst which can additionally be doped with further metals, including nickel, is used as catalyst. As noble metal, it is possible to use platinum in admixture with other noble metals. The noble metals are present in the catalyst as metals and the dopant metals are present in the form of salts.

DE 39 28 329 describes a process for preparing chlorine-substituted aromatic amines from the corresponding nitro compounds. The catalyst used in this process comprises activated carbon as support onto which platinum and a further metal, in particular nickel, are applied.

EP 595 124 describes a process for preparing chlorine-substituted aromatic amines from the corresponding nitro compounds. The catalyst used comprises platinum and nickel on activated carbon. Here, platinum is firstly applied to the activated carbon and reduced and nickel is then applied to the support in the form of a salt. The nickel is present as hydroxide in this catalyst.

EP 768 917 describes a catalyst for preparing carboxylic acid salts. This comprises an anchor metal, for example platinum, which is partly embedded in an alkali-resistant support and is at least partly coated with a catalytically active base metal, for example nickel, by electroless coating. In this catalyst, the two metals are present as separate phases on the support.

U.S. Pat. No. 4,185,036 describes a process for the hydrogenation of mixtures of nitroaromatics. The catalysts used comprise platinum and, if appropriate, a further metal, for example nickel, on activated carbon. The further metal is present in the form of the oxide or hydroxide on the support.

DE 199 11 865 and DE 196 36 214 describe processes for the hydrogenation of dinitrotoluene. The catalysts used comprise iridium and at least one dopant element, for example nickel or platinum.

WO 03/39743 describes a process for preparing TDA using a hydrogenation catalyst comprising platinum, a further noble metal and a base metal.

WO 05/037768 describes catalysts and processes for the hydrogenation of dinitrotoluene to toluenediamine. The catalysts comprise platinum and nickel, with the two metals being present in the form of an alloy on the support.

US 2004/0199017 describes a process for the hydrogenation of dinitrotoluene to toluenediamine using a catalyst comprising nickel, palladium and a third metal selected from the group consisting of zinc, cadmium, copper and silver in an amount of from 0.01 to 10% by weight, based on the weight of the support.

A continuing objective in the hydrogenation of DNT to TDA is to increase the yield further and in particular to improve the selectivity of the process in order to suppress secondary reactions which lead to the formation of high molecular weight by-products or to the formation of low boilers. Furthermore, the catalyst should also be stable at relatively high reaction temperatures and not permit any deterioration in the selectivity of the process.

To operate an economical process, the production and work-up of the catalyst have to be very advantageous. The production of the catalyst becomes cheaper when it is carried out in very few production steps. The work-up of exhausted noble metal catalysts becomes cheaper when the proportion of additional base metal components is very low.

It was therefore an object of the invention to provide catalysts for the hydrogenation of aromatic nitro compounds to the corresponding amines, in particular of DNT to TDA, which lead to a higher yield and selectivity of the process and are inexpensive to produce and work up.

This object has surprisingly been able to be achieved by the use of hydrogenation catalysts comprising nickel, palladium and an additional element selected from the group consisting of cobalt, iron, vanadium, manganese, chromium, platinum, iridium, gold, bismuth, molybdenum, selenium, tellurium, tin and antimony on a support in the hydrogenation of aromatic nitro compounds to the corresponding amines.

The invention accordingly provides a process for preparing aromatic amines by catalytic hydrogenation of the corresponding nitro compounds, in particular for preparing toluenediamine by hydrogenation of dinitrotoluene, wherein hydrogenation catalysts in which a mixture of nickel, palladium and an additional element selected from the group consisting of cobalt, iron, vanadium, manganese, chromium, platinum, iridium, gold, bismuth, molybdenum, selenium, tellurium, tin and antimony is present as active component on a support are used.

The invention further provides catalysts for the preparation of aromatic amines by catalytic hydrogenation of the corresponding nitro compounds, in particular for the preparation of toluenediamine by hydrogenation of dinitrotoluene, which comprise a mixture of nickel, palladium and an additional element selected from the group consisting of cobalt, iron, vanadium, manganese, chromium, platinum, iridium, gold, bismuth, molybdenum, selenium, tellurium, tin and antimony as active component on a support.

The invention further provides for the use of hydrogenation catalysts comprising a mixture of nickel, palladium and an additional element selected from the group consisting of cobalt, iron, vanadium, manganese, chromium, platinum, iridium, gold, bismuth, molybdenum, selenium, tellurium, tin and antimony as active component on a support for preparing aromatic amines by catalytic hydrogenation of the corresponding nitro compounds, in particular for preparing toluenediamine by hydrogenation of dinitrotoluene.

The additional element is preferably selected from the group consisting of cobalt and iron.

The metal particles are usually polycrystalline and can be characterized by means of a high resolution TEM (FEG-TEM: field emission gun-transmission electron microscopy).

As supports for the catalysts, it is possible to use the known materials customary for this purpose. Preference is given to using activated carbon, carbon black, graphite or metal oxides, preferably hydrothermally stable metal oxides such as ZrO₂, TiO₂. In the case of graphite, HSAG (high surface area graphite) having a surface area of from 50 to 300 m²/g is particularly preferred. Particular preference is given to activated carbon, in particular physically or chemically activated carbon, or carbon black, e.g. acetylene black.

The hydrogenation catalysts according to the invention preferably comprise from 5 to 30% by weight, in particular from 10 to 20% by weight, of nickel, from 0.01 to 20% by weight, in particular from 0.01 to 5% by weight, of palladium and from 0.1 to 20% by weight, in particular from 0.01 to 5% by weight, of the additional element, in each case based on the weight of the catalyst.

When carrying out the hydrogenation process of the invention, the catalyst according to the invention is preferably used in an amount of from 0.01 to 10% by weight, particularly preferably from 0.01 to 5% by weight, in particular from 0.2 to 3% by weight, based on the reaction mixture.

The catalyst is usually introduced into the reactor in the reduced, preferably reduced and passivated, state. For the purposes of the invention, the reduced and passivated state of the catalyst means that the catalyst is activated after its production but the active sites are then passivated for safety reasons, for example by passing oxygen or carbon dioxide over the catalyst. As an alternative, the catalyst can be removed from the production reactor under an inert atmosphere or in a relatively nonflammable solvent and stabilized, for example in water, TDA/water or higher alcohols such as butanol or ethylene glycol.

The process of the invention can be carried out continuously or batchwise using customary reactors and customary process parameters such as pressure and temperature.

The process for preparing aromatic amines, in particular TDA, using the catalysts according to the invention is preferably carried out at pressures in the range from 5 to 100 bar, particularly preferably from 10 to 40 bar, in particular from 20 to 25 bar.

The process for preparing aromatic amines, in particular DNT, using the catalysts according to the invention is preferably carried out at a temperature in the range from 80 to 250° C., particularly preferably in the range from 100 to 220° C. and in particular in the range from 160 to 200° C.

The hydrogenation is usually carried out in the form of a continuous suspension hydrogenation in customary and suitable reactors. Reactors used are, for example, stirred vessels or loop reactors, for example jet loop reactors, known as loop Venturi reactors, or loop reactors having internal flow reversal, as described in WO 00/35852. To separate off the catalysts from the discharged reaction mixture, it is possible to use, for example, crossflow filters. Such a process is described, for example, in WO 03/66571.

The amines formed in the hydrogenation are taken off continuously or discontinuously during the hydrogenation and subjected to a work-up, for example an after-treatment by distillation.

In the process of the invention, preference is given to using aromatic nitro compounds having one or more nitro groups and from 6 to 18 carbon atoms, for example nitrobenzenes such as o-, m-, p-nitrobenzene, 1,3-dinitrobenzene, nitrotoluenes such as 2,4-, 2,6-dinitrotoluene, 2,4,6-trinitrotoluene, nitroxylenes such as 1,2-dimethyl-3-, 1,2-dimethyl-4-, 1,4-dimethyl-2-, 1,3-dimethyl-2-, 2,4-dimethyl-1- and 1,3-dimethyl-5-nitrobenzene, nitronaphthalenes such as 1-, 2-nitronaphthalene, 1,5- and 1,8-dinitro-naphthalene, chloronitrobenzenes such as 2-chloro-1,3-, 1-chloro-2,4-dinitrobenzene, o-, m-, p-chloronitrobenzene, 1,2-dichloro-4-, 1,4-dichloro-2-, 2,4-dichloro-1- and 1,2-dichloro-3-nitrobenzene, chloronitrotoluenes such as 4-chloro-2-, 4-chloro-3-, 2-chloro-4- and 2-chloro-6-nitrotoluene, nitroanilines such as o-, m-, p-nitroaniline; nitro alcohols such as tris(hydroxymethyl)nitromethane, 2-nitro-2-methyl-, 2-nitro-2-ethyl-1,3-propanediol, 2-nitro-1-butanol and 2-nitro-2-methyl-1-propanol and also any mixtures of two or more of the nitro compounds mentioned.

Preference is given to hydrogenating aromatic nitro compounds, preferably mononitrobenzene, methylnitrobenzene or methylnitrotoluene and in particular 2,4-dinitrotoluene or its industrial mixtures with 2,6-dinitrotoluene, with these mixtures preferably comprising up to 35 percent by weight, based on the total mixture, of 2,6-dinitrotoluene together with proportions of from 1 to 5 percent of vicinal DNT and from 0.5 to 1.5% of 2,5- and 3,5-dinitrotoluene, to the corresponding amines by the process of the invention.

The catalysts of the invention can be used in a hydrogenation process in which the aromatic nitro compound is used in pure form, as a mixture with the corresponding diamine and/or polyamine, as a mixture with the corresponding diamine and/or polyamine and water, as a mixture with the corresponding diamine and/or polyamine, water and an alcoholic solvent or as a mixture with the corresponding diamine and/or polyamine, water, an alcoholic solvent and a catalyst-reactivating additive, with it also being possible in each case to use mixtures of two or more of the abovementioned nitro compounds, the corresponding amine compounds, the alcoholic solvent and the catalyst-reactivating additive.

If a mixture as described above is used, the ratio of amine compound to water is preferably in the range from 10:1 to 1:10, particularly preferably in the range from 4:1 to 1:1, and the ratio of the amine/water mixture to at least one alcoholic solvent is preferably from 1000:1 to 1:1, particularly preferably from 50:1 to 5:1.

To suppress secondary reactions, the process is preferably carried out so that the catalyst is operated at its loading limit. This can, for example, be controlled by means of the amount of nitro compound introduced, the amount of catalyst in the reaction mixture, the temperature or the pressure.

For the purposes of the present invention, the loading limit of the catalyst is the amount of the hydrogenatable nitrogen- and oxygen-comprising groups which can be hydrogenated by the catalyst under given pressure and temperature conditions. The nitrogen- and oxygen-comprising groups can be not only nitro groups but also nitroso groups and nitrosamine groups.

The catalysts of the invention are produced by, for example, placing the support in a reaction vessel and bringing it into contact with an aqueous solution of the palladium and nickel salts together with the additional element. The amount of water used for dissolving the salts should be such that a kneadable paste is formed. The water is preferably used in an amount of from 100 to 200% by weight of the support. As metal salts, use is made of, in particular, nitrates or chlorides, with nitrates being preferred because they are less corrosive. The paste is mixed and the water is then evaporated at a low pressure and temperatures in the range from 50 to 100° C., for example in a rotary evaporator or an oven. For safety reasons, evaporation can be carried out in a stream of nitrogen. When using chlorides as metal salts, the fixing of the metals on the support can be effected by reduction by means of hydrogen. However, corrosion can occur here. The metals are therefore preferably fixed under alkaline conditions. This is achieved, in particular, by adding an aqueous solution of alkali metal carbonates and subsequently washing the support free of anions. As an alternative, the metals can also be precipitated onto the support from a supernatant solution under alkaline conditions, in particular at a pH in the range from 8 to 9. The support is then dried, preferably as described above, and reduced by means of hydrogen. This can occur, for example, in a rotary bulb furnace. Before the catalyst is removed from the furnace, it is passivated, for example under an inert gas such as nitrogen comprising traces of air, preferably not more than 10% by volume.

The novel hydrogenation catalysts prepared by this process preferably comprise from 0.5 to 5% by weight of palladium, from 10 to 20% by weight of nickel and from 0.5 to 5% by weight of the additional element.

In another embodiment of the production of the hydrogenation catalysts according to the invention, the catalysts are reduced by addition of salts having a reducing action, for example ammonium carboxylates or alkali metal carboxylates, e.g. ammonium formate or sodium formate. For this purpose, the support is suspended in water and the solutions of the metal salts are added at the same time or after suspension has been carried out. Metal salts used are in particular nitrates or chlorides, with nitrates being preferred because they are less corrosive. The salts having a reducing action are added to this solution and the suspension is heated, for example by boiling under reflux. The catalysts are subsequently washed until free of anions and filtered, for example by means of a filter press or a centrifuge, and used as a moist paste.

The novel hydrogenation catalysts produced by this process preferably comprise from 0.5 to 5% by weight of palladium, from 10 to 20% by weight of nickel and from 0.5 to 5% by weight of the additional element.

The use of the catalysts of the invention makes it possible to carry out the hydrogenation of DNT to TDA at temperatures in the range from 120 to 250° C., in particular from 120 to 200° C., at which the selectivity of the reaction deteriorates greatly when conventional catalysts are used. An increase in the reaction temperature is advantageous since the solubilities of the individual components are higher and the reaction rate also increases with temperature. The STY (space-time yield) can thus be increased, as long as the energy of reaction can be safely removed.

The invention is illustrated in more detail by the following examples.

### EXAMPLE 1 (COMPARISON)

An activated carbon support Norit® SX+ support was suspended in water to form a 10% strength suspension. Pd(II) nitrate for 1.0% by weight of palladium, based on the weight of the catalyst, Ni(II) nitrate hexahydrate for 15% by weight of nickel and zinc(II) nitrate hexahydrate for 1.0% by weight of zinc were added and the mixture was refluxed with ammonium formate for 2 hours. The catalyst obtained in this way was washed until free of nitrate.

The catalyst obtained in this way will be referred to as catalyst 1.

The catalyst obtained in this way had a content of 0.92% by weight of palladium, 14% by weight of nickel and 0.96% by weight of zinc.

### EXAMPLE 2

The procedure of example 1 was repeated, but 1.0% by weight of palladium, 15% by weight of nickel and 1.0% by weight of tin were added. The catalyst obtained in this way will be referred to as catalyst 2. The catalyst obtained in this way had a content of 0.80% by weight of palladium, 13% by weight of nickel and 0.93% by weight of tin.

### EXAMPLE 3

The procedure of example 1 was repeated, but 1.0% by weight of palladium, 15% b_{y} weight of nickel and 1.0% by weight of gold were added. The catalyst obtained in this way will be referred to as catalyst 3. The catalyst obtained in this way had a content of 0.98% by weight of palladium, 10% by weight of nickel and 0.96% by weight of gold.

### EXAMPLE 4

The procedure of example 1 was repeated, but 1.0% by weight of palladium, 15% by weight of nickel and 1.0% by weight of iron were added. The catalyst obtained in this way will be referred to as catalyst 4. The catalyst obtained in this way had a content of 0.74% by weight of palladium, 8% by weight of nickel and 0.83% by weight of iron.

### EXAMPLE 5

The procedure of example 1 was repeated, but 1.0% by weight of palladium, 15% b_{y} weight of nickel and 1.0% by weight of cobalt were added. The catalyst obtained in this way will be referred to as catalyst 5. The catalyst obtained in this way had a content of 0.85% by weight of palladium, 13% by weight of nickel and 0.81% by weight of cobalt.

Hydrogenation of DNT to TDA

The hydrogenation of DNT to TDA was carried out in a continuous 300 ml stirred vessel, and the catalyst was mechanically retained in the reactor.

The catalyst was suspended in water and introduced into the reactor (amount of catalyst=1 to 2% by weight of the liquid volume of the reactor) and brought to a temperature of 180° C. Under a hydrogen pressure of 25 bar, DNT was fed in continuously as a melt in such an amount that a space-time yield of 150-600 kg_{TDA}/m³·h was established. Samples were analyzed by means of gas chromatography: the TDA yield, formation of high boilers and low boilers were monitored.

The catalysts, the composition thereof and the results are shown in table 1.

| Example | | | TDA |
|---|---|---|---|
| # | CAT | | Sel. % |
| 1 | 0.92%Pd—14%Ni—0.96Zn/C | Ca. 200 | 98.77 |
| (comparison) | | Ca 700 | 98.97 |
| 2 | 0.80%Pd—13%Ni—0.93Sn/C | Ca 400 | 98.47 |
| | | Ca 700 | 98.51 |
| 3 | 0.98%Pd—10%Ni—0.96Au/C | Ca 200 | 98.62 |
| | | Ca 700 | 98.83 |
| 4 | 0.74%Pd—8%Ni—0.83Fe/C | Ca 400 | 98.79 |
| | | Ca 700 | 99.04 |
| 5 | 0.85%Pd—13%Ni—0.81Co/C | Ca 400 | 98.82 |
| | | Ca 700 | 99.07 |

The examples show that the catalysts according to the invention lead to a high selectivity of the process. Particularly when iron and cobalt are used, very good results are achieved.

1. A process for preparing aromatic amines by catalytic hydrogenation of the corresponding nitro compounds, in particular for preparing toluenediamine by hydrogenation of dinitrotoluene, wherein hydrogenation catalysts in which a mixture of nickel, palladium and an additional element selected from the group consisting of cobalt, iron, vanadium, manganese, chromium, platinum, iridium, gold, bismuth, molybdenum, selenium, tellurium, tin and antimony is present as active component on a support are used.

2. The process according to claim 1, wherein the additional metal is selected from the group consisting of cobalt and iron.

3. The process according to claim 1, wherein the support is selected from the group consisting of activated carbon, carbon black, graphite and metal oxides.

4. The process according to claim 1, wherein the catalyst comprises from 5 to 30% by weight of nickel, from 0.01 to 20% by weight of palladium and 0.01-20% by weight of the additional element, in each case based on the weight of the catalyst.

5. The process according to claim 1, wherein the catalyst comprises from 10 to 20% by weight of nickel, from 0.5 to 5% by weight of palladium and 0.5-5% by weight of the additional element, in each case based on the weight of the catalyst.

6. The process according to claim 1, wherein the catalyst is used in an amount of from 0.01 to 5% by weight, based on the reaction mixture.

7. A catalyst for the preparation of aromatic amines by catalytic hydrogenation of the corresponding nitro compounds, in particular for the preparation of toluenediamine by hydrogenation of dinitrotoluene, which comprises a mixture of nickel, palladium and an additional element selected from the group consisting of cobalt, iron, vanadium, manganese, chromium, platinum, iridium, gold, bismuth, molybdenum, selenium, tellurium, tin and antimony as active component on a support.

8. The catalyst according to claim 7, wherein the additional element is selected from the group consisting of cobalt and iron.

9. The catalyst according to claim 7, wherein the support is selected from the group consisting of activated carbon, carbon black, graphite and metal oxides.

10. The catalyst according to claim 7 which comprises from 10 to 20% by weight of nickel, from 0.5 to 20% by weight of palladium and 0.5-5% by weight of the additional element, in each case based on the weight of the catalyst.

11. A process for producing hydrogenation catalysts according to claim 7, which comprises the steps
a) mixing of a support with the aqueous solutions of at least one palladium salt, at least one nickel salt and at least one salt of cobalt, iron, vanadium, manganese, chromium, platinum, iridium, gold, bismuth, molybdenum, selenium, tellurium, tin or antimony,
b) evaporation of the water,
c) addition of a water-soluble basic compound,
d) reduction of the catalyst formed by means of hydrogen.

12. The process according to claim 11, wherein steps a) and c) are carried out simultaneously.

13. A process for producing hydrogenation catalysts according to claim 7, which comprises the steps
a) suspension of the support in water, addition of aqueous solutions of at least one palladium salt, at least one nickel salt and at least one salt of cobalt, iron, vanadium, manganese, chromium, platinum, iridium, gold, bismuth, molybdenum, selenium, tellurium, tin or antimony,
b) addition of a water-soluble compound having a reducing action,
c) washing and filtration of the catalyst.

14. The use of catalysts according to claim 7 for the hydrogenation of dinitrotoluene.
